(19) United States
(12) Patent Application Publication
(10) Pub. No.: US 2005/0106725 A1
(43) Pub. Date: May 19, 2005

(54) Method of reducing cell differentiation

(76) Inventors: Sean P. Palecek, Madison, WI (US); Juan J. de Pablo, Madison, WI (US); Lin Ji, Madison, WI (US)

Correspondence Address: QUARLES & BRADY LLP, FIRSTAR PLAZA, ONE SOUTH PINCKNEY STREET, P.O. BOX 2113 SUITE 600, MADISON, WI, 53701-2113, US

(21) Appl. No.: 10/717,677

(22) Filed: Nov. 19, 2003

(51) Int. Cl. C12N 5/08

(52) U.S. Cl. 435/366

(57) ABSTRACT

The present invention relates to methods and compositions for culturing embryonic stem (ES) cells. The methods relate to growing the cells in culture on a flexible solid porous matrix, suitably without conditioned media or fibroblast feeder cells and applying an effective amount of periodic strain to stretch the flexible matrix, such that the cells proliferate and exhibit reduced differentiation. These methods in part facilitate a reduction in cross-species contamination for ES cells used in medical applications.

### STATEMENT REGARDING FEDERALLY SPONSORED RESEARCH OR DEVELOPMENT

This invention was made with United States government support awarded by the following agency: NAVY/ONR N66001-02-C-8051. The United States has certain rights in this invention.

### CROSS-REFERENCE TO RELATED APPLICATIONS

Not applicable.

### BACKGROUND OF THE INVENTION

Embryonic stem cells (ES cells) are a type of uncommitted, certainly pluripotent, and probably totipotent stem cell isolated from embryonic tissue. ES cells are particularly useful in research because they have the ability to indefinitely proliferate in vitro in an undifferentiated state; maintain a normal karyotype through prolonged culture; and maintain the potential to differentiate even after prolonged culture. However, embryonic stem cells are difficult to maintain in culture because they tend to spontaneously differentiate (i.e., acquire specialized structural and/or functional features). Stem cells differentiate as a result of many factors, including growth factors, extracellular matrix molecules and components, environmental stressors and direct cell-to-cell interactions. The specific agents that may enhance or direct stem cell differentiation along a particular path, however, maybe difficult to predict.

Currently, to grow human embryonic stem (ES) cells without cell differentiation, requires that the cells be cultured using mouse embryonic fibroblasts (MEFs) or extracellular matrix with conditioned media from mouse feeder cells. For example, Keller et al., have described that for ongoing cultures, cells from the inner mass of blastocysts are typically grown on a layer of mouse embryonic fibroblast “feeder” cells to preserve their undifferentiated phenotype and proliferabilty (See, Keller et al., Curr Opin Cell Biol (1995); 7:862-69). Also, related techniques for isolating stable cultures of human embryonic stem cells have recently been described by Thomson et al., in U.S. Pat. No. 5,843,780 and J. Thomson et al., 282 Science 1145-1147 (1998). The disclosure of these publications and of all other publications referred to herein are incorporated by reference as if fully set forth below.

Although stem cell cultures based on murine cell culture “feeder cell” technology are widely used, they are not highly desired. Non-species specific feeder cell technology reduces the value of stem cell cultures due to the foreign nature of the source of the feeder cell. This is true for a number of reasons including the fact that such non-species specific feeder cells contain both foreign cells and foreign growth factors. Further, it is believed that the use of non-species specific feeder cells in combination with different but desirable cultured cells cannot provide the optimum growth conditions as species specific derived feeder cells or conditioned media. The issue of cross-species contamination is particularly relevant to agricultural animals, endangered species, laboratory animals and non-human primate cells. Still further, non-human feeder cell technology reduces the value of human derived stem cell cultures. This is true for number of reasons including the fact that such non-human feeder cells contain both non-human cells and non-human growth factors. Also, it is believe that the use of non-human feeder cells in combination with human cultured cells cannot provide the optimum growth conditions as human derived feeder cells.

It would be desirable to have a method of culturing embryonic stem cells that results in reduced cell differentiation without the use of conditioned media and in the absence of mouse embryonic fibroblast feeder cells. This would ensure that embryonic stem cells are not subject to cross-species contamination with biological materials from another species and are grown or maintained with optimal growth conditions.

### BRIEF SUMMARY OF THE INVENTION

The present invention is summarized as providing methods and compositions for culturing embryonic stem (ES) cells. The methods are directed to growing the cells in culture on a flexible solid porous matrix, suitably without exposure to fibroblast feeder cells or conditioned media. Then an effective amount of periodic strain is applied to stretch the flexible matrix, such that the cells proliferate and exhibit reduced differentiation.

In one aspect the invention prevents cross-species contamination by the presence of biological material (i.e., cells, organelles, metabolic products, peptides, antibodies, etc.) from another species.

In one aspect the invention provides that the cells are grown on Matrigel™ using BioFlex® untreated culture plates.

In one aspect the invention provides that the cells are human embryonic stem cells.

In another aspect the invention provides that the strain is mechanically produced.

In another aspect the invention provides that the flexible matrix is stretched using vacuum pressure.

In another aspect the invention provides that the strain exerted on the flexible matrix is at least about 5% strain.

In another aspect the invention provides that the mechanical strain applied to the matrix surface is produced by oscillatory stretching motions.

In another aspect the invention provides that the flexible matrix undergoes at least about 6 stretches per minute.

In yet another aspect the invention provides for a culture, wherein ES cells are grown on a flexible solid porous matrix, suitably without conditioned media and in the absence of fibroblast feeder cells, and an effective amount of periodic strain is applied to stretch the flexible matrix, such that the cells exhibit reduced differentiation.

Unless otherwise defined, all technical and scientific terms used herein have the same meaning as commonly understood by one of ordinary skill in the art to which this invention belongs. Although suitable methods and materials for the practice or testing of the present invention are described below, other methods and materials similar or equivalent to those described herein, which are well known in the art, can also be used.

Other objects, advantages and features of the present invention will become apparent from the following specification taken in conjunction with the accompanying drawings.

### BRIEF DESCRIPTION OF THE SEVERAL VIEWS OF THE DRAWINGS

FIG. 1 is a schematic of a Bioflex® culture plate illustrating how forces are applied to cause stain: (A) top view of a Bioflex® culture plate; and (B) cross-section view of a single well.

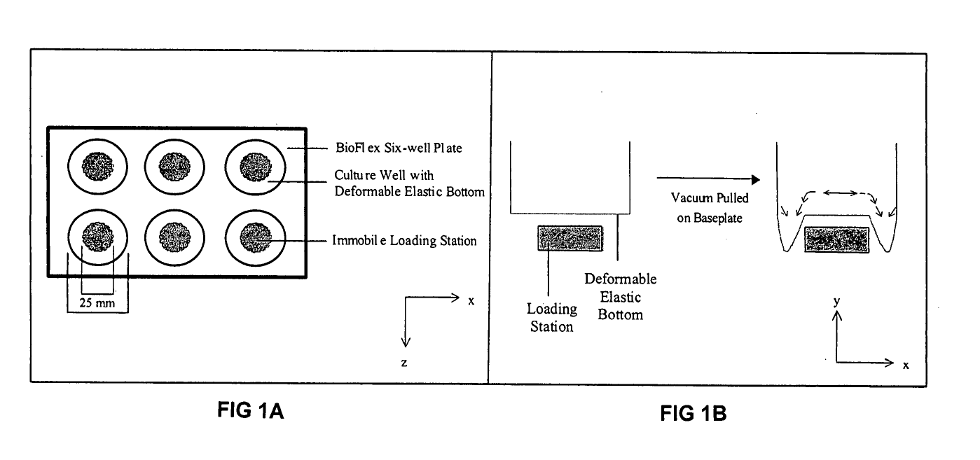

FIG. 2 is a phase contrast image of ES cells grown 14 days under 10% strain vs. ES cells not grown under stain, such that (A) the ES cells grown under strain are undifferentiated, smaller, and less spread out and (B) the absence of differentiation in ES cells is characterized by a high level of OCT4 expression.

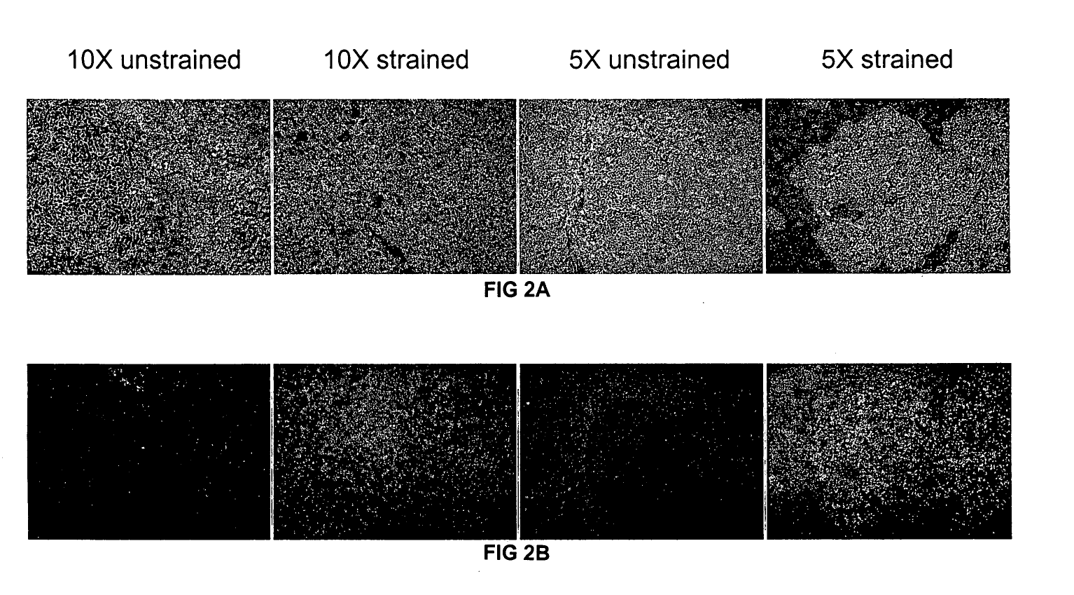

### DETAILED DESCRIPTION OF THE INVENTION

In many applications, a strong need for culture technology capable of growing and maintaining stable or useful cultures of stem cells has been a highly desired end. The present invention provides methods and compositions for culturing embryonic stem (ES) cells with reduced differentiation. The methods involve growing ES cells in culture on a flexible solid porous matrix, suitably without exposure to fibroblast feeder cells or conditioned media. Then an effective amount of periodic strain is applied to stretch the flexible matrix, such that the cells proliferate and exhibit reduced differentiation.

In practicing the invention, it is suitable for the cells to be grown on Matrigel™ using BioFlex® untreated culture plates. It is envisioned that other less expensive alternatives to Matrigel™ may be used in the invention. Other non-limiting substrate alternatives may include collagen, hyaluronic acid, gelatin material, ProNectin, Laminin, vitronectin, fibronectin, elastin, collagen, entactin, a proteoglycan, or mixtures thereof, which mediate cell adherence to the substrate. Likewise, other types of suitable flexible culture plates, amenable to being stretched, elongated or deformed, such as rubber or gel-matrices are also encompassed by the present invention.

Also, in accordance with the invention, it is envisioned that if cross-species contamination does not appear to be a concern, exposure of the cells to fibroblast feeder cell culture or conditioned media, may be used to co-stimulate the cells in conjunction with other stem cell cultures.

Then, an effective amount of periodic strain is applied to the surface of the flexible matrix, stretching the matrix, either simultaneously with or subsequent to cell growth, such that the cells proliferate and exhibit reduced differentiation. Suitably the addition of strain eliminates differentiation over a specific time period. It is encompassed that the strain applied to the flexible matrix maybe used to physically or mechanically stretch the matrix using vacuum pressure. Suitably, it has been identified that the flexible matrix undergoes at least about 6 stretches per minute.

Furthermore, it is believed that several commercial systems may be employed to facilitate cellular stretching in accordance with the present invention. For example, the Flexercell® Tension Plus™ (FX-4000T™)(Dunn Labortechnik GmBH) is particularly suitable to facilitate stretching of the matrix. Flexercell® Tension Plus™ is a computer-driven system that simulates biological strain conditions using ‘vacuum’ to deform cells cultured on a flexible, matrix-bonded growth surface of BioFlex® or Flex® series culture plates. This system provides a strain component for dynamically culturing a cells in vitro and possibly inducing biochemical changes in response to the strain.

The Flexercell® Tension Plus™ system is capable of applying a defined, controlled, static, or cyclic deformation or stretch to growing cells in vitro. It has been determined that the vacuum used to deform a flexible-bottom culture plate can yield up to 30% substrate or flexible matrix elongation.

The flexible matrix is stretched using the Flexcell system and under the appropriate conditions described below, the level of cell differentiation is assessed using a variety of methods. In accordance with the invention, the OCT4 gene marker is used to measure ES cell differentiation, as described in the examples below. Specifically, OCT4 is an embryonic gene transcription factor, that plays an role in control of developmental pluripotency, so that when OCT4 gene activity is repressed in pluripotent stem cells differentiation occurs. (See, Pesce, et al., (1998) Mech. Dev., 71:89). As such, OCT4 is a useful marker of pluripotency, since undifferentiated ES cells express OCT4 consistent with their pluripotent nature while ES cell differentiation is associated with signal loss. Accordingly, in the present application OCT4 is used a marker to assess the developmental potential of cells through conventional immunostaining techniques.

It is further noted that other markers maybe used to detect cellular differentiation. Generally in an undifferentiated state, ES cells are alkaline phosphatase-positive, express immunological markers characteristic of embryonic stem and embryonic germ cells, express telomerase and retain the capacity for extended self-renewal.

For example, it has been determined that growing the cells on a flexible surface without strain results in a slight decrease in differentiation versus growth on a standard polystyrene surface. However, the addition of 10% strain resulting in approximately 6 stretches per minute was able to eliminate differentiation over a two-week growth period with suitable media changes and without cell passaging. As a comparison, during the same two-week growth period the cells grown under the same conditions on a polystyrene surface showed about 25% differentiation. Therefore, growing the cells on a flexible matrix combined with applying an sufficient amount of strain was able to effectively eliminate cell differentiation over the two-week period of time without media changes and without cell passaging.

In another embodiment of the invention, a culture is provided comprising ES cells grown on a flexible solid porous matrix suitably without conditioned media and in the absence of fibroblast feeder cells, wherein an effective amount of periodic strain is applied to stretch the flexible matrix, such that the cells exhibit reduced differentiation. It is noted that suitable culture plate growth surfaces may include Matrigel™ using BioFlex® untreated culture plates and other alternatives as described hereinabove. Thus, one aspect of the invention is to ensure that ES cells are not contaminated by the presence of biological material (i.e., cells, organelles, metabolic products, peptides, antibodies, etc.) from another species, reducing the possibility of an antigenic immune response.

The invention will be further described in the following examples, which do not limit the scope of the invention defined by the claims.

### EXAMPLES

Growth:

Human embryonic stem (HES) cells were grown on Matrigel™ using BioFlex® untreated culture plates from FlexCell International Corp. (Lower Burrell, Pa.).

Making Matrigel™ Plate:

To prepare a Matrigel™ plate, a tube of Matrigel™ stock (2 mg) was taken directly from the −20° C. freezer. Matrigel™ was obtained from Becton Dickinson, San Jose, Calif. The Matrigel™ pellet was immediately resuspended in 6 ml ice cold DMEM/F12. All chunks in the mixture were eliminated through vigorous pipetting. A 1 ml aliquot of the Matrigel™ mixture was added to each well of the 6-well BioFlex untreated culture plate. The plate was maintained at room temperature for one hour or overnight at 4° C. before use.

Making Conditioned Media (CM):

In accordance with the invention, it is desired that when human ES cells are cultured that conditioned media not be used, since one aspect of practicing the methods of present invention, is to eliminate cross-species contamination of the ES cell culture. However, it is encompassed that CM maybe used to culture ES cells. Thus, to prepare CM, a plate was coated with 0.1% gelatin solution, 10 ml to a T75 flask. After the plate was coated, it was incubated overnight in a 37° C., humidified incubator with 5% CO₂ for 24 hours prior to plating irradiated MEFs. To a T75 flask, 15 ml of irradiated MEF cells were added in MEF medium (90% DMEM, 10% FBS, and 1% MEM non-essential amino acids solution) with a concentration at 2.12×10⁵ cells/ml. The flask was incubated overnight. The MEF medium was aspirated away and 20 ml HES medium without bFGF (80% DMEM/F12 medium, 20% Knockout Serum Replacement, 1% L-glutamine solution, and 0.1 mM MEM non-essential amino acids solution) was added to the flask. The flask was again incubated overnight. The medium was collected and 20 ml of fresh HES medium without the bFGF was added to the flask. Every day for up to 2 weeks, the medium was collected. Then bFGF as added to the collected medium to 4 ng/ml final concentration before putting on the cells.

Splitting Human Embryonic Stem Cell Culture:

In order to split HES cell culture, the medium from HES cell culture plate was aspirated. Collagenase splitting medium (1 ml at 1 mg/ml in DMEM/F12) was added to each well in a 6-well plate. The plate was incubated in a 37° C., humidified incubator with 5% CO₂, for about 3-5 min. It was confirmed that the edges of the colonies were separating from the surface of the plate by microscope inspection. The tip of a glass 5 ml pipet was used to scrape the colonies off the surface of the plate for a 6 well plate. The colony suspension was transferred into a sterile 15 ml conical tube. The cells were gently pipetted up and down a few times in the tube, to further break up the colonies. The cells were pelleted by centrifugation at 1000 rpm for 5 min and the supernatant was aspirated. The cell pellet was washed by adding about 3 ml of HES medium to the 15 ml conical tube and the pellet was gently reconstituted in the HES medium. The mixture was then centrifuged at 1000 rpm for 5 min. The supernatant was aspirated off and the cell pellet was washed by adding 3 ml of HES medium to the 15 ml conical and gently reconstituting the pellet in medium. The mixture was centrifuged at 1000 rpm for 5 min. While the HES cells were spinning for the second time, the Matrigel solution was aspirated away from the BioFlex® culture plate. The plate was then washed once with 2 ml per well of 1× calcium and magnesium-free PBS solution and the mixture was centrifuged at 1000 rpm for 5 min. The supernatant was aspirated away from the HES cell pellet after the second spin. A sufficient volume of medium was added to form the desired number of cells for the split. The medium was mixed well. The PBS solution was aspirated away from the wells. The HES cells were evenly dispensed among the desired number of wells by adding them dropwise to each well. After the HES cells were plated, the plates were returned to the incubator and moved in several quick, short, back-and-forth and side-to-side motions. The cells were then incubated in a humidified 37° C. incubator with 5% CO₂. The culture was then refreshed once per day with medium.

Stretching:

In order to stretch the cells, fresh growth medium (4 ml or 5 ml) was added to each well of the BioFlex culture plate. The Flexercell® Tension Plus™, FX-4000T™ ((Flexcell International, McKeesport, Pa.), was then placed on the cells to produce cyclic stretch using vacuum. The plate was carefully inserted into the gasket. The baseplate was connected to FX-4000T™. The program was loaded on to the computer and a suitable configuration was chosen. The following parameters were created: the regimen with the waveform shape, the desired minimum and maximum % elongation, frequency, regimen time, and length/duration of cycles. The pump was turned on. Then the suitable regimen was assigned and run.

Differentiation:

Cell dfferentiation was then viewed everyday with an inverted culture microscope. Moreover, in the present invention to observe the level of cell differentiation for the HES cells grown on stretched flexible matrix, an inverted culture microscope was utilized. It is noted that HES cells are best viewed at a lower objective, such as 2.5×, where several colonies can be observed at once, as well as at a higher objective, such as 10×, where individual colonies and cell morphology can be observed.

Furthermore, as indicated above, for ES cells to be applicable for a variety of medical applications, the cells must retain the capacity for unlimited proliferation and differentiation. Any differentiation will limit their use in downstream applications. Accordingly, it is encompassed that the state of cell differentiation may be assessed by the presence of stem cell surface markers OCT4 and SSEA-4 using immunofluorescence microscopy (Xu, C., et al., (2001) Nat Biotechnol 19: 971-974, incorporated by reference herein in its entirety). Alkaline phosphatase activity can also be used to verify the undifferentiated state of ES cells (Pera, M. F., et al., (2000) J Cell Sci 113 (Pt 1): 5-10). Furthermore, flow cytometric analysis using an anti-CD34 antibody and a fluorescent secondary antibody may be used to identify certain fractions of stem cell populations that are capable of differentiating (Kaufman, D. S., et al., (2001) Proc Natl Acad Sci USA 98: 10716-10721).

Immunostaining of OCT4 on the BioFlex Culture Plate:

After a sufficient amount of stretching, the cells were fixed on the BioFlex culture plate with 4% paraformaldahyde for 10 to 15 min. Optionally, a blocking solution was added containing 5% normal serum or milk in 1×KPBS (0.154M NaCl, 0.04M K2HPO4, 0.01M KH2PO4) at 4° C. for 1 hr. Then, 1 ml KPBS+0.4% Triton was added to each well. Also, OCT4 primary antibody (1:100˜1:200) was added and the cells were incubated at room temperature for 1 hr or at 4° C. for overnight. The cells were washed with 1×KPBS 5 times and 1 ml of 1×KPBS+0.4% Triton was added. The secondary antibody (FITC) (1:500 to about 1:1000) was added and the cells were again incubated at room temperature for 1 hr. The cells were washed with 1×KPBS 3 times. The cells were then photographed. Undifferentiated ES cells were characterized by OCT4 protein expression.

Flow Cytometry:

In order to measure the viability of the cells, 2 ml of collagenase was added per well to the cells for 10 minutes at 37° C. The cells were scraped from the wells, resuspended, and spun down at 1000 rpm for 5 min. The pellet was resuspended in 2 ml Trypsin/EDTA+2% chick serum and incubated in a 37° C. waterbath for 10 min. The resuspended mixture was diluted with medium and spun down. The pellet was resuspended in FACs buffer (2% FBS, 0.1% NaN3 in 1× calcium and magnesium-free PBS solution) and the cells were passed thru 80 or 40 micron mesh filter into a 50 ml conical tube. The cells were counted, if necessary, and transferred to a 15 ml tube. The cells were spun down at 1000 rpm for 5 min. The cells were washed 1-2 times in 2 ml FACs buffer and spun down. The cells were resuspended accordingly in FACs buffer to provide a density at ˜1×10⁶ cells/ml and 100 ul was aliquoted per each tube. The primary antibody (SSEA-4) was added to the tube and vortexed lightly. The cell mixture was incubated for 30 min at room temperature or 45 min on ice. FACs buffer, 2 ml, was added to each tube, vortexed, and spun down. The supernatant was discarded, leaving ˜100 ul buffer with the pellet. The secondary antibody dilution was added to the cell pellet, which was vortexed, and incubated for 30 min at room temperature or 45 min on ice in the dark. FACs buffer, 2 ml, was added to each tube, which was vortexed, and spun down. The pellet was resuspended in 300-500 ul of FACs buffer. The resuspended cells were put on ice and analyzed by fluorescence-activated cell sorting (FACS) on a FACS Calibur system (Becton Dickinson). Before analysis to stain whether the cells were live or dead, 5 ul of Propidium Iodide (1 mg/ml in PBS) was added per tube.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A method of culturing embryonic stem (ES) cells with reduced differentiation comprising:
a) growing the cells in culture on a flexible solid porous matrix without conditioned media and in the absence of fibroblast feeder cells; and
b) applying an effective amount of periodic strain to stretch the flexible matrix, such that the cells proliferate and exhibit reduced differentiation.

2. The method of claim 1 wherein the cells are mammalian embryonic stem cells.

3. The method of claim 1 wherein the cells are human embryonic stem cells.

4. The method of claim 1 wherein the cell differentiation is eliminated.

5. The method of claim 1 wherein the cells are grown on Matrigel™ using BioFlex® untreated culture plates.

6. The method of claim 1 wherein the cells are grown without the presence of cross-species biological material.

7. The method of claim 1 wherein the flexible matrix is Matrigel™.

8. The method of claim 1 wherein the strain is mechanically produced.

9. The method of claim 1 wherein the flexible matrix is stretched using vacuum pressure.

10. The method of claim 1 wherein the strain exerted on the flexible matrix is at least about 5%.

11. The method of claim 1 wherein the flexible matrix undergoes at least about 6 stretches per minute.

12. The method of claim 1 wherein the mechanical strain is from oscillatory stretching of the flexible matrix surface.

13. A culture comprising embryonic stem (ES) cells grown on a flexible solid porous matrix without conditioned media or fibroblast feeder cells, wherein an effective amount of periodic strain is applied to stretch the flexible matrix, such that the cells proliferate and exhibit reduced differentiation.

14. The method of claim 13 wherein the cells are mammalian embryonic stem cells.

15. The method of claim 13 wherein the cells are human embryonic stem cells.

16. The method of claim 13 wherein the cell differentiation is eliminated.

17. The method of claim 13 wherein the cells are grown on Matrigel™ using BioFlex® untreated culture plates.

18. The method of claim 13 wherein the cells are grown without the presence of cross-species biological material.

19. The method of claim 13 wherein the flexible solid porous matrix is Matrigel™.

20. The method of claim 13 wherein the strain is mechanically produced.

21. The method of claim 13 wherein the flexible matrix is stretched using vacuum pressure.

22. The method of claim 13 wherein the mechanical strain is from oscillatory stretching of the flexible matrix surface.

23. The method of claim 13 wherein the strain exerted on the flexible matrix is at least about 5%.

24. The method of claim 13 wherein the flexible matrix undergoes at least about 6 stretches per minute.
